(19) United States
(12) Patent Application Publication
(10) Pub. No.: US 2013/0197230 A1
(43) Pub. Date: Aug. 1, 2013

(54) METHODS OF PREPARING QUINOLINE DERIVATIVES

(71) Applicants: Jo Ann Wilson, San Francisco, CA (US); Sharique Zuberi, Folsom, CA (US); Sriram Naganathan, San Jose, CA (US); Erick Goldman, Concord, CA (US); James Kanter, Hayward, CA (US)

(72) Inventors: Jo Ann Wilson, San Francisco, CA (US); Sharique Zuberi, Folsom, CA (US); Sriram Naganathan, San Jose, CA (US); Erick Goldman, Concord, CA (US); James Kanter, Hayward, CA (US)

(21) Appl. No.: 13/129,183

(22) PCT Filed: Nov. 13, 2009
(86) PCT No.: PCT/US2009/064341

§ 371 Date: Jul. 24, 2012

Related U.S. Application Data
(60) Provisional application No. 61/199,088, filed on Nov. 13, 2008.

(51) Int. Cl. C07D 215/233

(52) U.S. Cl. CPC C07D 215/233
USPC 546/153

(57) ABSTRACT

Methods of preparing compounds of formula i(1):

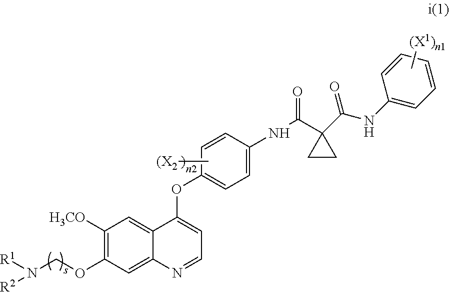

or a pharmaceutically acceptable salt thereof, wherein:
R¹ and R² join together with the nitrogen atom to which they are attached form a 6 membered heterocycloalkyl group;
X¹ is H, Br, Cl or F;
X² is H, Br, Cl or F;
s is 2-6;
n1 is 1-2; and
n2 is 1-2.

### CROSS REFERENCE TO RELATED APPLICATIONS

This application claims the benefit of U.S. Provisional Patent Application No. 61/199,088, filed Nov. 13, 2008, the contents of which is incorporated herein by reference in its entirety.

### BACKGROUND OF THE INVENTION

1. Field of the Invention

This disclosure relates to methods of preparing compounds useful for modulating protein kinase enzymatic activity. More specifically, this disclosure relates to methods of preparing compounds useful for modulating cellular activities such as proliferation, differentiation, programmed cell death, migration and chemoinvasion.

2. Summary of the Related Art

Improvements in the specificity of agents used to treat cancer is of considerable interest because of the therapeutic benefits which would be realized if the side effects associated with the administration of these agents could be reduced. Traditionally, dramatic improvements in the treatment of cancer are associated with identification of therapeutic agents acting through novel mechanisms.

Protein kinases are enzymes that catalyze the phosphorylation of proteins, in particular, hydroxy groups on tyrosine, serine and threonine residues of proteins. The consequences of this seemingly simple activity are staggering; cell differentiation and proliferation; i.e., virtually all aspects of cell life in one-way or another depend on protein kinase activity. Furthermore, abnormal protein kinase activity has been related to a host of disorders, ranging from relatively non-life threatening diseases such as psoriasis to extremely virulent diseases such as glioblastoma (brain cancer).

Therapeutic use of kinase modulation can relate to oncological indications. For example, modulation of protein kinase activity for the treatment of cancer has been demonstrated successfully with the FDA approval of GleevecG) (imatinib mesylate, produced by Novartis Pharmaceutical Corporation of East Hanover, N.J.) for the treatment of Chronic Myeloid Leukemia (CML) and gastrointestinal stroma cancers (GIST). Gleevec is a c-Kit and Abl kinase inhibitor.

Modulation (particularly inhibition) of cell proliferation and angiogenesis, two key cellular processes needed for tumor growth and survival (Matter A. Drug Disc Technol 20016, 1005-1024), is an attractive goal for development of small-molecule drugs. Anti-angiogenic therapy represents a potentially important approach for the treatment of solid tumors and other diseases associated with dysregulated vascularization, including ischemic coronary artery disease, diabetic retinopathy, psoriasis and rheumatoid arthritis. As well, cell antiproliferative agents are desirable to slow or stop the growth of tumors.

A target of interest for small-molecule modulation, with respect to antiangiogenic and antiproliferative activity is c-Met. The kinase, c-Met, is the prototypic member of a subfamily of heterodimeric receptor tyrosine kinases (RTKs) which include Met, Ron and Sea. Expression of c-Met occurs in a wide variety of cell types including epithelial, endothelial and mesenchymal cells where activation of the receptor induces cell migration, invasion, proliferation and other biological activities associated with “invasive cell growth.” As such, signal transduction through c-Met receptor activation is responsible for many of the characteristics of tumor cells.

The endogenous ligand for c-Met is the hepatocyte growth factor (HGF), a potent inducer of angiogenisis, also known as “scatter factor” (SF). Binding of HGF to c-Met induces activation of the receptor via autophosphorylation resulting in an increase of receptor dependent signaling, which promotes cell growth and invasion. Anti-HGF antibodies or HGF antagonists have been shown to inhibit tumor metastasis in vivo (See: Maulik et al Cytokine & Growth Factor Reviews 2002 13, 41-59).

Tumor growth progression requires the recruitment of new blood vessels into the tumor from preexisting vessels as well as invasion, adhesion and proliferation of malignant cells. Accordingly, c-Met overexpression has been demonstrated on a wide variety of tumor types including breast, colon, renal, lung, squamous cell myeloid leukemia, hemangiomas, melanomas, astrocytomas, and glioblastomas. Additionally activating mutations in the kinase domain of c-Met have been identified in hereditary and sporadic renal papilloma and squamous cell carcinoma. (See: Maulik et al Cytokine & growth Factor reviews 2002 13, 41-59; Longati et al Curr Drug Targets 2001, 2, 41-55; Funakoshi et al Clinica Chimica Acta 2003 1-23). Thus modulation of c-Met is desirable as a means to treat cancer and cancer-related disease.

Accordingly, there is a need for new methods of making compounds that are protein kinase modulators.

### SUMMARY OF THE INVENTION

In one aspect, the disclosure relates to methods of preparing compounds of formula i(1):

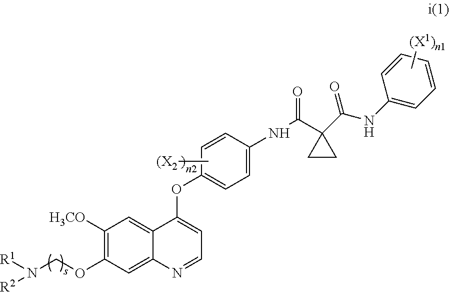

or a pharmaceutically acceptable salt thereof, wherein:

R¹ and R² join together with the nitrogen atom to which they are attached form a 6 membered heterocycloalkyl group;

X¹ is H, Br, Cl or F;

X² is H, Br, Cl or F;

s is 2-6;

n1 is 1-2; and

n2 is 1-2.

Intermediates useful in preparing the above compounds are also disclosed.

The compounds of formula i(1) are useful as protein kinase modulators, and they inhibit c-Met.

There are many different aspects and embodiments of the disclosure described hereinbelow, and each aspect and each embodiment is non-limiting in regard to the scope of the disclosure. The terms “aspects” and “embodiments” are meant to be non-limiting regardless of where the terms “aspect” or “embodiment” appears in this specification. The transitional term “comprising” as used herein, which is synonymous with “including,” “containing,” or “characterized by,” is inclusive or open-ended and does not exclude additional, unrecited elements.

### DETAILED DESCRIPTION OF THE INVENTION

Aspect (1) of this disclosure relates to a method of preparing a compound of formula i(1):

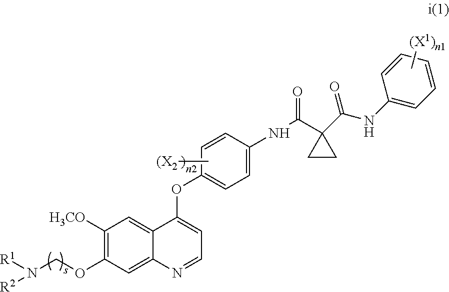

or a pharmaceutically acceptable salt thereof, wherein:

R¹ and R² join together with the nitrogen atom to which they are attached form a 6 membered heterocycloalkyl;

X¹ is H, Br, Cl or F;

X² is H, Br, Cl or F;

s is 2-6;

n1 is 1-2; and

n2 is 1-2,

the method comprising:
contacting the compound of formula h(1) with reactant z(1) to yield the compound of formula i(1):

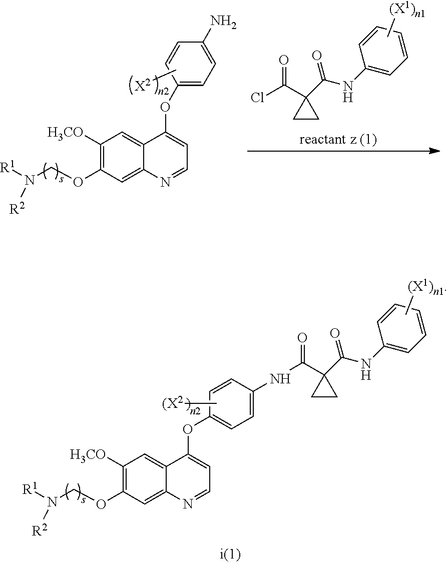

The reaction in Aspect (1) of this disclosure is advantageously carried out under suitable reaction conditions. Non-limiting examples of suitable reaction conditions in Aspect (1) include using basic conditions. Non-limiting examples of basic conditions that can be used in Aspect (1) of this disclosure include the use of inorganic bases, such as aqueous KOH, NaOH, K₂CO₃, Na₂CO₃, K₃PO₄, Na₃PO₄, K₂HPO₄, Na₂HPO₄, and the like, or mixtures thereof. Other non-limiting examples of suitable reaction conditions in Aspect (1) include using suitable solvents. Non-limiting examples of suitable solvents that can be used in Aspect (1) of this disclosure include water miscible solvents, such as THF, acetone, ethanol, and the like, or mixtures thereof. Other non-limiting examples of suitable solvents that can be used in Aspect (1) of this disclosure include water immiscible solvents such as MTBE, dichloromethane (DCM), isopropopyl acetate (iPAc), toluene, and the like, or mixtures thereof. Other non-limiting examples of suitable reaction conditions in Aspect (1) include using suitable temperatures. Suitable temperatures that may be used for the reaction in Aspect (1) include a temperature at a range of from about 7° C. to about 30° C., or alternatively, at a range of from about 10° C. to about 26° C., or alternatively, at a range of from about 12° C. to about 21° C. The product formed in Aspect (1) is in the free base form and this free base form may be converted into a pharmaceutically acceptable salt thereof, by methods known in the art. In one example, the compound of formula i(1) can be converted to its bis-maleate salt by the addition of maleic acid and a suitable solvent. In another example, the compound of formula i(1) can be converted to its bis-phosphate salt by the addition of phosphoric acid and a suitable solvent.

Utilities of the compound of Formula I are further described in WO 2005/030140 A2.

### Embodiments of Aspect (1) (Part A)

In another embodiment of Aspect (1), X¹ is Cl or F.

In another embodiment of Aspect (1), X² is Cl or F.

In another embodiment of Aspect (1), X¹ is F.

In another embodiment of Aspect (1), X² is F.

In another embodiment of Aspect (1), X¹ is H.

In another embodiment of Aspect (1), X² is H.

In another embodiment of Aspect (1), n1 is 1.

In another embodiment of Aspect (1), n2 is 1.

In another embodiment of Aspect (1), n1 is 2.

In another embodiment of Aspect (1), n2 is 2.

In another embodiment of Aspect (1), s is 2.

In another embodiment of Aspect (1), s is 3.

In another embodiment of Aspect (1), s is 4.

In another embodiment of Aspect (1), s is 5.

In another embodiment of Aspect (1), s is 6.

In another embodiment of Aspect (1), R¹ and R² join together with the nitrogen atom to which they are attached form piperidinyl, piperazinyl or morpholinyl.

In another embodiment of Aspect (1), R¹ and R² join together with the nitrogen atom to which they are attached form morpholinyl.

All compounds of formula i(1) for Aspect (1) disclosed above include any of the disclosed alternative embodiments in Part A for each of X¹, X², n1, n2, or s, in combination with any other of the disclosed alternative embodiments in Part A for each of X¹, X², n1, n2, or s, as well as a pharmaceutically acceptable salt of any such combination.

### Embodiments of Aspect (1) Part B

In another embodiment of Aspect (1), n1 and n2 are each 1.

In another embodiment of Aspect (1), n1 and n2 are each 2.

In another embodiment of Aspect (1), n1 is 1; and n2 is 2.

In another embodiment of Aspect (1), n1 is 2 and n2 is 1.

In another embodiment of Aspect (1), X¹ is H; and X² is F.

In another embodiment of Aspect (1), X¹ is F; and X² is H.

In another embodiment of Aspect (1), X¹ and X² are each H.

In another embodiment of Aspect (1), X¹ and X² are each F.

In another embodiment of Aspect (1), X¹ is Cl; and X² is H.

In another embodiment of Aspect (1), X¹ is H; and X² is Cl.

In another embodiment of Aspect (1), X¹ and X² are each Cl.

In another embodiment of Aspect (1), X¹ is Cl; and X² is F.

In another embodiment of Aspect (1), X¹ is F; and X² is Cl.

In another embodiment of Aspect (1), s is 3; and R¹ and R² join together with the nitrogen atom to which they are attached form morpholinyl.

In embodiment (C) of Aspect (1), the compound of formula h(1) can be made by reducing a compound of formula g(1) to yield the compound of formula h(1):

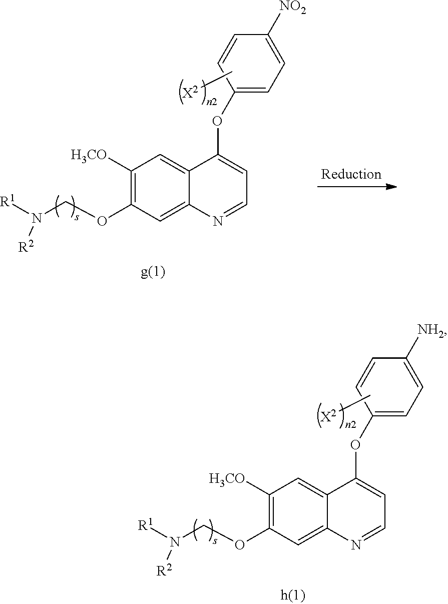

wherein each of R¹, R², X², S and n2 are as defined in Aspect (1), or as in any of the embodiments of Aspect (1) (Part A), of this disclosure.

The reaction in embodiment (C) of Aspect (1) of this disclosure is advantageously carried out under suitable reaction conditions. Non-limiting examples of suitable reaction conditions in embodiment (C) of Aspect (1) include reducing the compound of formula g(1) to the compound of formula h(1) in the presence of a catalyst. Non-limiting examples of such catalysts that can be used in embodiment (C) of Aspect (1) include platinum group metals, and the like. Non-limiting examples of catalysts that are platinum group metals include palladium, platinum, rhodium, ruthenium, and the like. Reduction of the compound of formula g(1) can also be carried out by non-catalytic reduction, such as with the use of dithionite, iron acid-acid, or tin-acid. In another embodiment of embodiment (C) of Aspect (1), the reaction is carried out in the presence of palladium on carbon (Pd/C). In another embodiment of embodiment (C) of Aspect (1), the reaction is carried out in the presence of about 5% to about 20% Pd/C. In another embodiment of embodiment (C) of Aspect (1), the reaction is carried out in the presence of about 7% to about 15% Pd/C in ethanol. In another embodiment of embodiment (C) of Aspect (1), the reaction is carried out in about 10% Pd/C in ethanol. In another embodiment of embodiment (C) of Aspect (1), the reduction using such catalyst is carried out by transfer hydrogenation in the presence of a hydrogen-transfer reagent, wherein the hydrogen-transfer reagent includes any hydrogen-transfer reagent known in the art which the skilled artisan would consider to be suitable for this reaction. In another embodiment of embodiment (C) of Aspect (1), the reduction is a transfer hydrogenation reaction carried out in the presence of an aqueous solution of formic acid and a formate such as ammonium formate, alkylammonium formate, or potassium formate. Other non-limiting examples of suitable reaction conditions that can be used in embodiment (C) of Aspect (1) include the use of suitable solvents for the reaction to take place in. Non-limiting examples of suitable solvents that can be used in embodiment (C) of Aspect (1) include THF, AcOH, ethanol (EtOH), EtOAc, and the like, or mixtures thereof. Other non-limiting examples of suitable reaction conditions that can be used in embodiment (C) of Aspect (1) include the use of hydrogen gas under a suitable pressure that can be used in the reaction. Suitable pressures that can be used in embodiment (C) of Aspect (1) include pressures ranging from about 10 psi to about 50 psi. Other non-limiting examples of suitable reaction conditions that can be used in embodiment (C) of Aspect (1) include the use of suitable temperatures that can be used in the reaction. Suitable temperature ranges for the reaction in embodiment (C) of Aspect (1) include temperatures that one skilled in the art would ordinarily use for this reaction. In another embodiment of embodiment (C) of Aspect (1), the reduction reaction can be carried out in the presence of about 10% palladium on carbon in a mixture of ethanol and water containing concentrated hydrochloric acid and pressurizing with hydrogen gas at approximately 40 psi. The reaction temperature can be at about ambient temperature. Once the reduction reaction is complete, any catalyst that may have been used can be removed, if so desired, by filtering the reaction mixture through a bed of Celite®. The reaction mixture can optionally be purified, for instance, by adding a basic solution, such as potassium carbonate, until the pH of the solution is from about 9 to about 11. The resulting suspension can than be stirred and the resultant solids can be collected by filtration under standard conditions.

In embodiment (D) of Aspect (1), the compound of formula g(1) can be made by reacting a compound of formula f(1) with reactant y(1) to yield the compound of g(1):

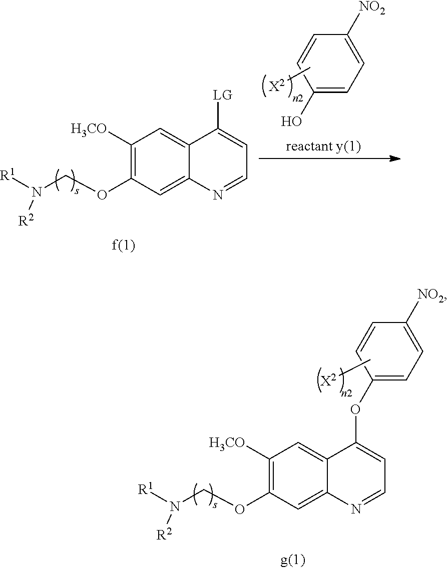

wherein LG represents a leaving group, and each of R¹, R², X², s and n2 are as defined in Aspect (1), or as in any of the embodiments of Aspect (1) (Part A), of this disclosure. A non-limiting example of a leaving group includes halo groups (such as Cl, Br or F). Various compounds of reactant y(1) are commercially available, such as 2-fluoro-4-nitrophenol. Also, the skilled artisan would be able to make any variation of reactant y(1) using commercially available starting materials and by using known techniques to modify these commercially available starting materials to come up with various compounds within the scope of reactant y(1).

The reaction in embodiment (D) of Aspect (1) of this disclosure is advantageously carried out under suitable reaction conditions. Non-limiting examples of suitable reaction conditions in embodiment (D) of Aspect (1) include using basic conditions, such as, for example, 2,6-dimethylpyridine (2,6-lutidine). Other non-limiting examples of suitable reaction conditions in embodiment (D) of Aspect (1) include using suitable reaction temperatures when the organic base is added, which can generally range from about 120° C. to about 180° C. In another embodiment, this reaction temperature can range from about 130° C. to about 160° C. In another embodiment, this reaction temperature can range from about 140° C. to about 150° C. Once the reaction is complete, a base, such as potassium carbonate, can be added to the reaction mixture to precipitate the solids, and then the precipitate can be collected by filtration under standard conditions.

In an alternative embodiment for embodiments (C) and (D) of Aspect 1, the compound of formula h(1) can be made by reacting the compound of formula f(1) with reactant u to yield the compound of formula h(1), wherein each of R¹, R², X², s and n2 are as defined in Aspect (1), or as in any of the embodiments of Aspect (1) (Part A), of this disclosure.

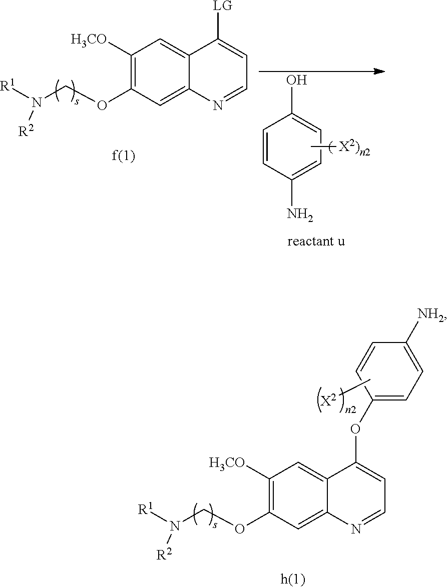

wherein LG represents a leaving group. A non-limiting example of a leaving group includes halo groups (such as Cl, Br or F). The alternative step for embodiments (C) and (D) of Aspect 1 above is advantageously carried out under suitable reaction conditions. Non-limiting examples of suitable reaction conditions in this alternative step for embodiments (C) and (D) of Aspect (1) include a suitable solvent. Non-limiting examples of a suitable solvents that can be used for this alternative step of embodiments (C) and (D) of Aspect 1 include polar solvents such as dimethylacetamide (DMA), dimethylsulfoxide (DMSO), dimethylformamide (DMF), ethyl acetate, N-methylpyrrolidone (NMP), propylene carbonate, and the like, or mixtures thereof. Other non-limiting examples of suitable reaction conditions in this alternative step for embodiments (C) and (D) of Aspect (1) include the use of a suitable base, such as non-nucleophilic base. Non-limiting examples of non-nucleophilic bases that can be used include lithium diisopropylamide, lithium tetramethylpiperidide and alkali metal alkoxides such as sodium tert-butoxide, potassium tert-butoxide, and the like, or mixtures thereof. Other non-limiting example of suitable reaction conditions include reaction temperatures ranging from about 75-120° C., or alternatively, 85-110° C., or alternatively, 95-100° C. The reaction mixture can then be cooled to below about 50° C. and additional base and reactant u can be added, and the reaction temperature can be increased again to the suitable reaction temperatures stated above to obtain additional yield with water-drown and isolation with filtration.

In embodiment (E) of Aspect (1), the compound of formula f(1) can be made by converting a compound of formula e(1) to the compound of formula f(1):

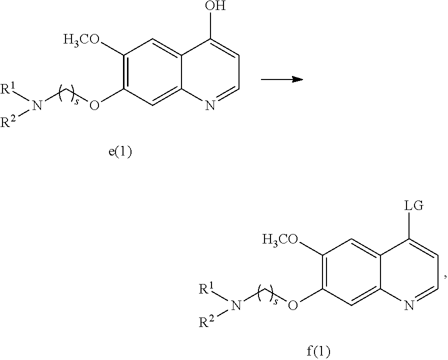

wherein LG represents a leaving group, and each of s, R¹ and R² are as defined in Aspect (1), or as in any of the embodiments of Aspect (1) (Part A), of this disclosure. A non-limiting example of a leaving group that could be used in embodiment (E) of Aspect (1) include halo groups (such as Cl, Br or F) that can be added by halogenating agents. Non-limiting examples of halogenating agents that can be used in embodiment (E) of Aspect (1) include chlorinating agents, such as SOCl₂, SO₂Cl₂, COCl₂, PCl₅, POCl₃, and the like.

The reaction in embodiment (E) of Aspect (1) of this disclosure is advantageously carried out under suitable reaction conditions. Non-limiting examples of suitable reaction conditions in embodiment (E) of Aspect (1) include the use of suitable solvents. Non-limiting example of suitable solvents that can be used in embodiment (E) of Aspect (1) during the halogenation of the compound of formula e(1) include a polar, aprotic solvent, such as ACN, DMF, and the like, or mixtures thereof. In other embodiments, the chlorination can be carried out using POCl₃ in acetonitrile, COCl₂ in DMF, or SOCl₂ in DMF. The addition of the chlorination agent is advantageously carried out at a temperature ranging from about 35° C. to about 75° C. In another embodiment, the addition of the chlorination agent can be carried out at a temperature ranging from about 45° C. to about 65° C. In another embodiment, the addition of the chlorination agent can be carried out at a temperature ranging from about 50° C. to about 60° C. After completion of the chlorination reaction, the mixture can be heated to reflux until the reaction is complete. The reaction mixture can then be filtered to remove solids, and the product in the filtrate can then be extracted using standard techniques.

In embodiment (F) of Aspect (1), the compound of formula e(1) can be made by converting a compound of formula d(1) to the compound of formula e(1) with an alkyl formate, such as methyl formate, ethyl formate, n-propyl formate, or i-propyl formate.

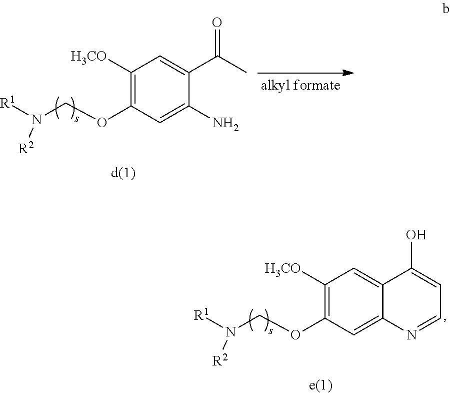

wherein each of s, R¹ and R² are as defined in Aspect (1), or as in any of the embodiments of Aspect (1) (Part A), of this disclosure.

The reaction in embodiment (F) of Aspect (1) of this disclosure is advantageously carried out under suitable reaction conditions. Non-limiting examples of suitable reaction conditions in embodiment (F) of Aspect (1) include the use of a suitable base. Non-limiting examples of a suitable base that can be used in embodiment (F) of Aspect (1) include strong bases, such as a sodium alkoxide (for instance, sodium ethoxide). Other non-limiting examples of suitable reaction conditions in embodiment (F) of Aspect (1) include the use of suitable solvents. Non-limiting examples of suitable solvents that can be used in embodiment (F) of Aspect (1) include alcohols in combination with esters, for example, ethanol and ethyl formate, and the like, or mixtures thereof. Other non-limiting examples of suitable reaction conditions in embodiment (F) of Aspect (1) include the use of suitable temperatures. The reaction is advantageously carried out at a suitable temperature ranging from about 30° C. to about 60° C. In another embodiment, this reaction can be carried out from about 40° C. to about 50° C. In another embodiment, this reaction can be carried out at about 44° C. After the reaction is complete, the product can be precipitated by adding any solvent that will cause the product to precipitate, for example, methyl-t-butyl ether (MTBE). The product can then be collected by filtration and optionally purified using standard techniques.

In embodiment (G) of Aspect (1), the compound of formula d(1) can be made by reducing a compound of formula c(1) to yield the compound of formula d(1):

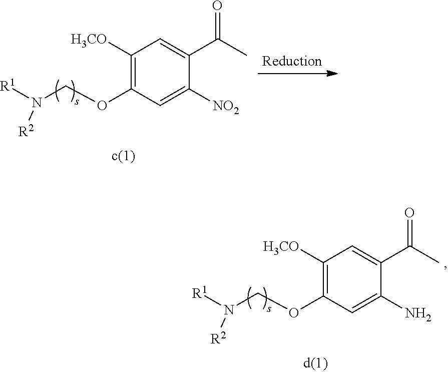

wherein each of s, R¹ and R² as defined in Aspect (1), or as in any of the embodiments of Aspect (1) (Part A), of this disclosure.

The reaction in embodiment (G) of Aspect (1) of this disclosure is advantageously carried out under suitable reaction conditions. Non-limiting examples of suitable reaction conditions in embodiment (G) of Aspect (1) include reducing the compound of formula c(1) to the compound of formula d(1) in the presence of a catalyst. Non-limiting examples of such catalysts that can be used in embodiment (G) of Aspect (1) include platinum group metals and the like. Non-limiting examples of catalysts that are platinum group metals include palladium, platinum, rhodium, ruthenium, and the like. Reduction of the compound of formula c(1) can also be carried out by non-catalytic reduction, such as with the use of dithionite, iron acid-acid, or tin-acid. In another embodiment of embodiment (G) of Aspect (1), the reaction is carried out in the presence of palladium on carbon (Pd/C). In another embodiment of embodiment (G) of Aspect (1), the reaction is carried out in the presence of about 5% to about 20% Pd/C. In another embodiment of embodiment (G) of Aspect (1), the reaction is carried out in the presence of about 7% to about 15% Pd/C in ethanol. In another embodiment of embodiment (G) of Aspect (1), the reaction is carried out in about 10% Pd/C in ethanol. In another embodiment of embodiment (G) of Aspect (1), the reduction is carried out by transfer hydrogenation in the presence of a hydrogen-transfer reagent, wherein the hydrogen-transfer reagent can be any hydrogen-transfer reagent known in the art which the skilled artisan would consider to be suitable for this reaction. In another embodiment of embodiment (G) of Aspect (1), the reduction is a transfer hydrogenation reaction carried out in the presence of an aqueous solution of formic acid and potassium formate. Other non-limiting examples of suitable reaction conditions that can be used in embodiment (G) of Aspect (1) include the use of suitable solvents for the reaction to take place in. Non-limiting examples of suitable solvents that can be used in embodiment (G) of Aspect (1) include tetrahydrofuran (THF), acetic acid (AcOH), ethanol (EtOH), EtOAc, isopropanol (IPA), and the like, or mixtures thereof. Other non-limiting examples of suitable reaction conditions that can be used in embodiment (G) of Aspect (1) include the use of suitable pressures that can be used in the reaction. Suitable pressures that can be used in embodiment (G) of Aspect (1) include pressures ranging from about 10 psi to about 50 psi.

In another embodiment of embodiment (G) of Aspect (1), the reduction is carried out by transfer hydrogenation in the presence of a hydrogen-transfer reagent, wherein the hydrogen-transfer reagent can be any hydrogen-transfer reagent known in the art which the skilled artisan would consider to be suitable for this reaction. In another embodiment of embodiment (G) of Aspect (1), the reduction is a transfer hydrogenation reaction carried out in the presence of an aqueous solution of formic acid and a formate such as potassium formate, ammonium formate or alkylammonium formate. Other non-limiting examples of suitable reaction conditions that can be used in embodiment (G) of Aspect (1) include the use of suitable temperatures that can be used in the reaction. Suitable temperature ranges for the reaction in embodiment (G) of Aspect (1) include temperatures that one skilled in the art would ordinarily use for this reaction. In another embodiment of embodiment (G) of Aspect (1), the reduction reaction can be carried out in the presence of about 10% palladium on carbon in a mixture of ethanol and water containing concentrated hydrochloric acid and pressurizing with hydrogen gas at approximately 40 psi. The reaction temperature can be at about ambient temperature. When the reaction is complete, the catalyst can be removed and the compound can be extracted using known techniques.

In embodiment (H) of Aspect (1), the compound of formula c(1) can be made by reacting a compound of formula b(1) with

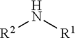

to yield the compound of formula c(1):

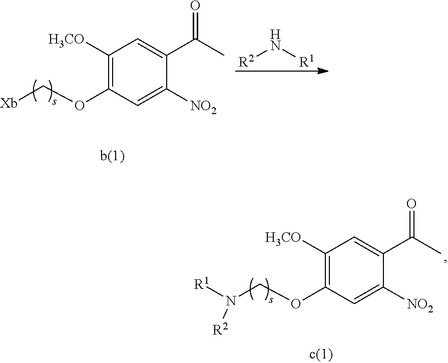

wherein Xb is Br or Cl; and each of s, R¹ and R² are as defined in Aspect (1), or as in any of the embodiments of Aspect (1) (Part A), of this disclosure.

The reaction in embodiment (H) of Aspect (1) of this disclosure is advantageously carried out under suitable reaction conditions. Non-limiting examples of suitable reaction conditions in embodiment (H) of Aspect (1) include using a phase transfer catalyst for the reaction to take place. Non-limiting examples of phase transfer catalysts that can be used in embodiment (H) of Aspect (1) include methyltributylammonium chloride, methyltriethylammonium chloride, tetrabutylammonium bromide, tetrabutylammonium chloride monohydrate, tetra-n-butylammonium bromide (Bu₄NBr), tetrabutylammonium hydrogen sulfate, tetrabutylammonium hydroxide, tetraethylammonium bromide, tetramethylammonium hydroxide, and the like. In another embodiment, the phase transfer catalyst used in embodiment (H) of Aspect (1) is tetra-n-butylammonium bromide (Bu₄NBr). Other non-limiting examples of suitable reaction conditions in embodiment (H) of Aspect (1) include using basic conditions for the reaction to take place. Non-limiting examples of bases that can be used in embodiment (H) of Aspect (1) include Cs₂CO₃, K₂CO₃, Na₂CO₃, and the like, or mixtures thereof. In another embodiment, the base that is used in embodiment (H) of Aspect (1) is K₂CO₃. Other non-limiting examples of suitable reaction conditions in embodiment (H) of Aspect (1) include using a suitable solvent for the reaction to take place. Non-limiting examples of solvents that can be used in embodiment (H) of Aspect (1) include dimethoxymethane (DME), THF, toluene, dichloromethane, and the like, or mixtures thereof. In another embodiment, the solvent that is used in embodiment (H) of Aspect (1) is toluene. In another embodiment of embodiment (H) of Aspect (1), the phase transfer catalyst is tetra-n-butylammonium bromide (Bu₄NBr), the solvent is toluene, and the base is K₂CO₃ (potassium carbonate). The product can be extracted by extraction techniques known in the art.

In embodiment (I) of Aspect (1) of this disclosure, the compound of formula b(1) can be made by reacting a compound of formula a(1) with HNO₃ to yield the compound of formula b(1):

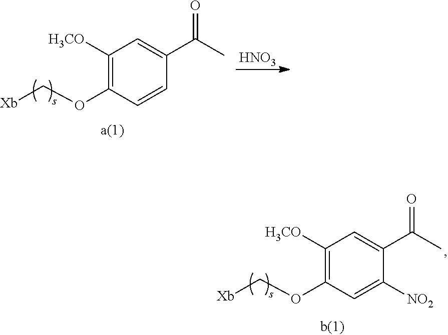

wherein Xb is Br or Cl; and each of s, R¹ and R² are as defined in Aspect (1), or as in any of the embodiments of Aspect (1) (Part A), of this disclosure.

The reaction in embodiment (I) of Aspect (1) of this disclosure is advantageously carried out under suitable reaction conditions. Non-limiting examples of suitable reaction conditions in embodiment (I) of Aspect (1) include reacting the compound of formula a(1) with HNO₃ in an acidic solution, such as H₂SO₄. Other non-limiting examples of suitable reaction conditions in embodiment (I) of Aspect (1) that can be used include conducting the reaction under temperatures in the range of from about 0° C. to about 15° C., or alternatively at a temperature in the range of from about 3° C. to about 10° C., or alternatively at a temperature in the range of from about 5° C. to about 10° C. The product b(1) can be separated by extraction techniques known in the art, for instance using methylene chloride, water and an aqueous potassium bicarbonate solution.

In embodiment (J) of Aspect (1) of this disclosure, reactant z(1) can be made by reacting reactant z(1a) with a chlorinating agent to yield reactant z(1):

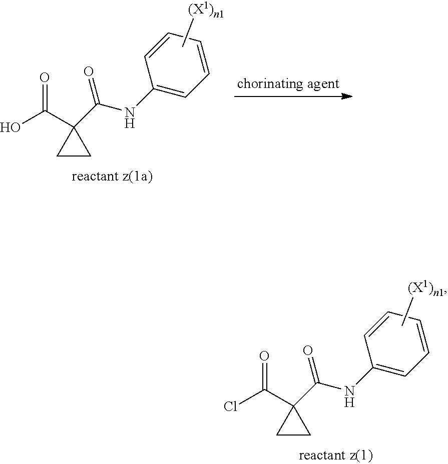

wherein X¹ is Br, Cl or F; and n1 is 1-2. Compounds of reactant z(1a) can be made according to the method described in Example 25 of WO 2005/030140 A2, and the skilled artisan would be able to make any necessary substitutions using commercially available starting materials to come up with various compounds within the scope of reactant z(1a). Example 25 in WO 2005/030140 A2 is incorporated herein by reference.

The reaction in embodiment (J) of Aspect (1) of this disclosure is advantageously carried out under suitable reaction conditions. Non-limiting examples of suitable reaction conditions in embodiment (J) of Aspect (1) include using a chlorinating agent such as POCl₃, oxalyl chloride, and the like. In another embodiment of embodiment (J) of Aspect (1), oxalyl chloride is used as a chlorinating agent. Non-limiting examples of suitable reaction conditions in embodiment (J) of Aspect (1) include carrying out the reaction at a temperature in the range from about 0° C. to about 15° C., or alternatively at a temperature in the range from about 3° C. to about 10° C., or alternatively at a temperature in the range from about 5° C. to about 10° C. Other non-limiting examples of suitable reaction conditions in embodiment (J) of Aspect include carrying out the reaction in a suitable solvent. Non-limiting examples of suitable solvents that can be used in embodiment (J) of Aspect (1) include polar, aprotic solvents such as halogenated hydrocarbons, i.e., dichloromethane, chloroform; or ethers, i.e., Et₂O, dioxane, tetrahydrofuran (THF) containing catalytic DMF, and the like, or mixtures thereof. The resulting solution containing reactant z(1) can be used, without further processing, to make the compound of formula i(1) in Aspect (1) of this disclosure.

In another embodiment of Aspect (1) of this disclosure, the compound of formula i(1) is of formula i(2):

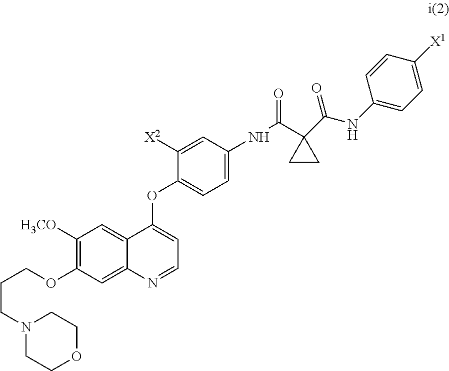

or a pharmaceutically acceptable salt thereof, wherein:

X¹ is H, Cl, Br or F; and

X² is H, Cl, Br or F. As mentioned above for the compound of formula i(1), the compound of formula i(2) can be in the free base form or it can converted to a pharmaceutically acceptable salt thereof. Accordingly, the compound of formula i(2) can be converted to its bis-maleate salt by the addition of maleic acid and a suitable solvent, and the compound of formula i(2) can be converted to its bis-phosphate salt by the addition of phosphoric acid and a suitable solvent.

In another embodiment of Aspect (1) of this disclosure, the compound is of formula i(2) wherein X¹ is F; and X² is F.

In another embodiment of Aspect (1) of embodiment (I) of this disclosure, the compound of formula a(1) is of formula a(2):

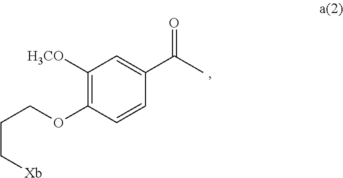

wherein Xb is Br or Cl; and

the compound of formula b(1) is of formula b(2):

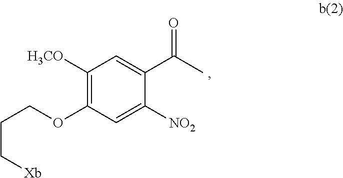

wherein Xb is Br or Cl.

In another embodiment of Aspect (1), embodiment (H) of this disclosure, the compound of formula b(1) is of formula b(2):

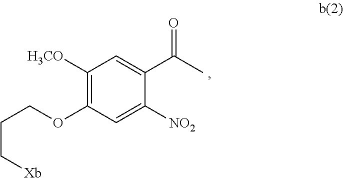

wherein Xb is Br or Cl;

the compound of formula c(1) is of formula c(2):

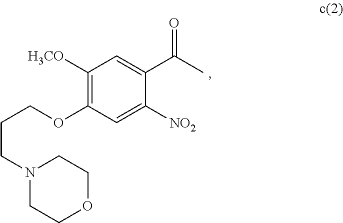

wherein Xb is Br or Cl; and

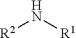

is morpholine.

In another embodiment of Aspect (1), embodiment (G) of this disclosure, the compound of formula c(1) is of formula c(2):

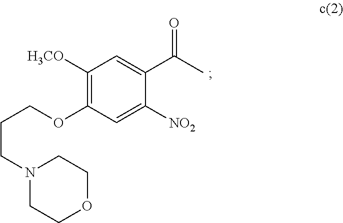 [SMILES: COc1cc(C(C)=O)c([N+](=O)[O-])cc1OCCCN1CCOCC1]

and the compound of formula d(1) is of formula d(2):

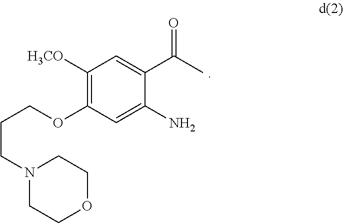 [SMILES: COc1cc(C(C)=O)c(N)cc1OCCCN1CCOCC1]

In another embodiment of Aspect (I), embodiment (F) of this disclosure, the compound of formula d(1) is of formula d(2):

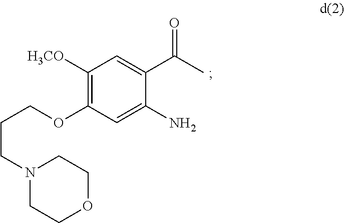 [SMILES: COc1cc(C(C)=O)c(N)cc1OCCCN1CCOCC1]

and

the compound of formula e(1) is of formula e(2):

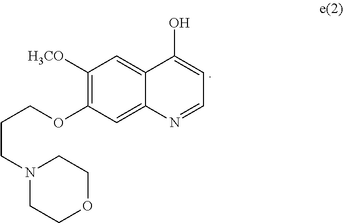 [SMILES: COc1cc2c(O)ccnc2cc1OCCCN1CCOCC1]

In another embodiment of Aspect (1), embodiment (E) of this disclosure, the compound of formula e(1) is of formula e(2):

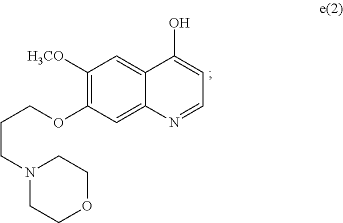 [SMILES: COc1cc2c(O)ccnc2cc1OCCCN1CCOCC1]

and the compound of formula f(1) is of formula f(2):

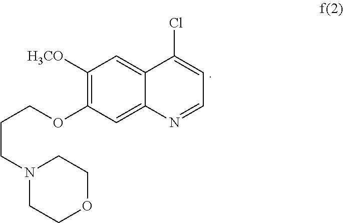 [SMILES: COc1cc2c(Cl)ccnc2cc1OCCCN1CCOCC1]

In another embodiment of Aspect (1), embodiment (D) of this disclosure, the compound of formula f(1) is of formula f(2):

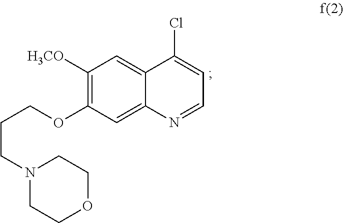 [SMILES: COc1cc2c(Cl)ccnc2cc1OCCCN1CCOCC1]

reactant y(1) is reactant (y)(2):

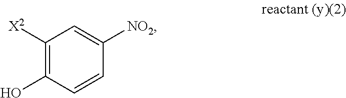

wherein X² is chloro or fluoro; and

the compound of formula g(1) is of formula g(2):

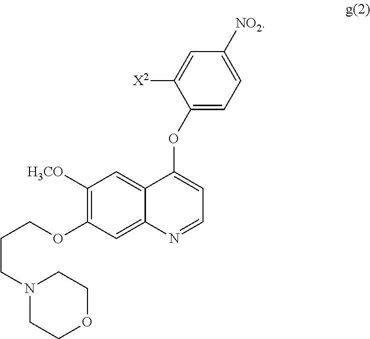

In another embodiment of Aspect (1), embodiment (C) of this disclosure, the compound of formula g(1) is of formula g(2):

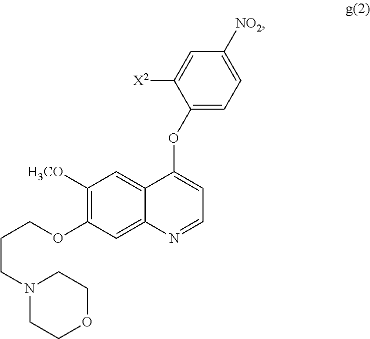

wherein X² chloro or fluoro; and

the compound of formula h(1) is of formula h(2):

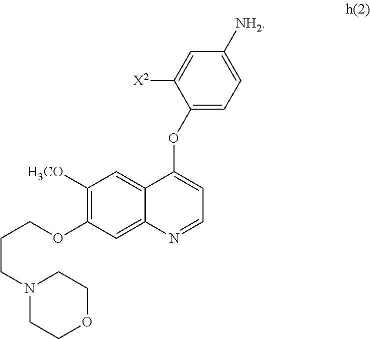

In another embodiment of the alternative embodiment for embodiments (C) and (D) of Aspect 1, the compound of formula f(1) is of formula f(3):

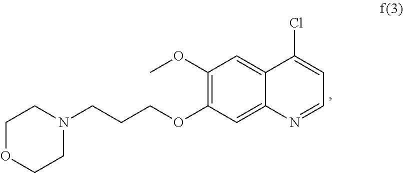 [SMILES: COc1cc2c(Cl)ccnc2cc1OCCCN1CCOCC1]

the compound of formula h(1) is of formula h(3):

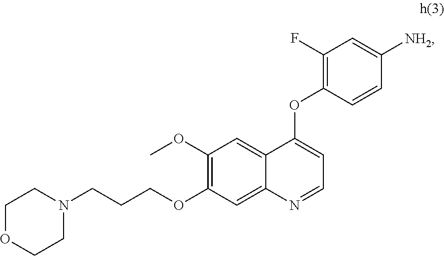 [SMILES: COc1cc2c(Oc3ccc(N)cc3F)ccnc2cc1OCCCN1CCOCC1]

and reactant u is reactant u2:

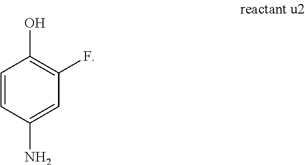

In another embodiment of Aspect (1) of this disclosure, the compound of formula h(1) is of formula h(2):

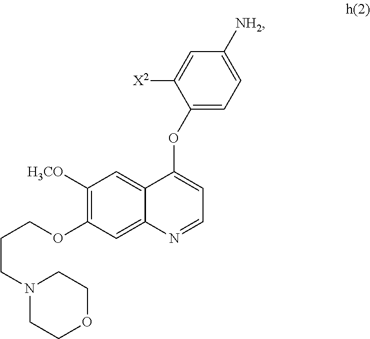

wherein X² is F;

reactant z(1) is reactant (z)(2):

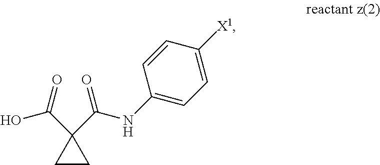

wherein X¹ is F; and

the compound of formula i(1) is of formula i(2):

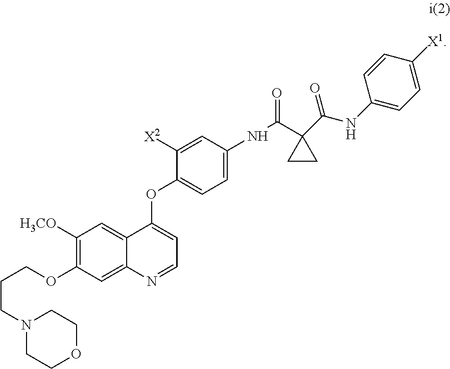

In another embodiment of Aspect (1) of this disclosure, X¹ and X² for each of formula h(2), i(2) and reactant z(2) are each selected from Cl or F. In another embodiment of Aspect (1) of this disclosure, X¹ and X² for each of formula h(2), i(2) and reactant z(2) are both F.

Aspect (2) of the disclosure relates to a method of preparing a compound of formula b(2):

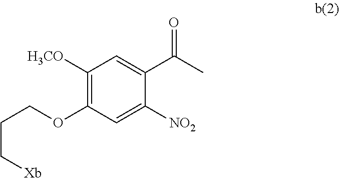

or a pharmaceutically acceptable salt thereof, the method comprising:

reacting a compound of the formula a(2):

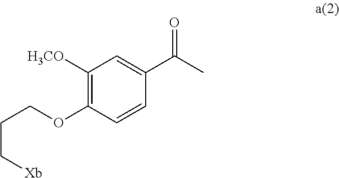

with HNO₃ in a suitable solvent to yield the compound of formula b(2):

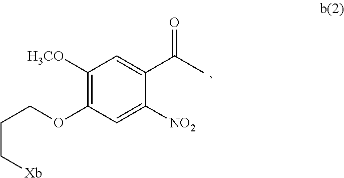

wherein Xa is Cl or Br; and Xb is Cl or Br. The compound of formula b(2) can be in its free base form. The reaction conditions that can be used in this aspect include any of the reaction conditions disclosed in embodiment (I) of Aspect (1).

Aspect (3) of the disclosure relates to a method of preparing a compound of formula c(1):

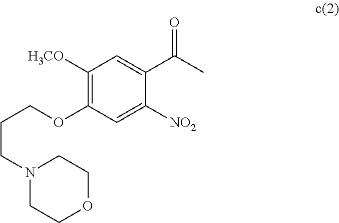 [SMILES: COc1cc(C(C)=O)c([N+](=O)[O-])cc1OCCCN1CCOCC1]

or a pharmaceutically acceptable salt thereof, the method comprising:

reacting a compound of formula b(2) with morpholine under basic conditions and with a phase transfer catalyst (such as tetra-n-butylammonium bromide (Bu₄NBr)) in a suitable solvent to yield the compound of formula c(2):

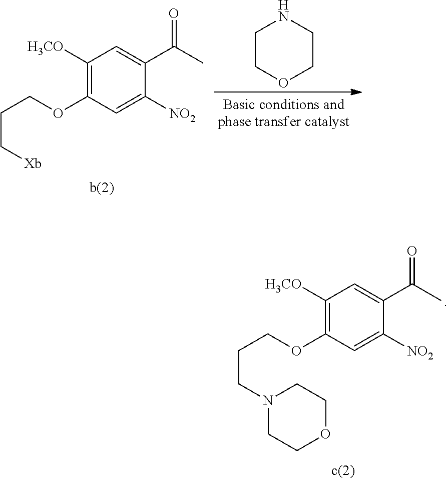

The compound of formula c(2) can be in its free base form or converted to a pharmaceutically acceptable salt thereof. The reaction conditions that can be used in this aspect include any of the reaction conditions disclosed in embodiment (H) of Aspect (1).

Aspect (4) of the disclosure relates to a method of preparing a compound of formula d(2):

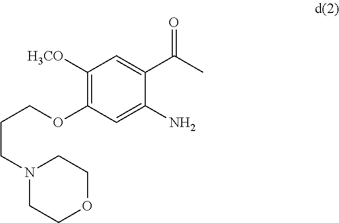 [SMILES: COc1cc(C(C)=O)c(N)cc1OCCCN1CCOCC1]

or a pharmaceutically acceptable salt thereof, the method comprising:

hydrogenating the compound of formula c(2) with a catalyst to yield the compound of formula d(2):

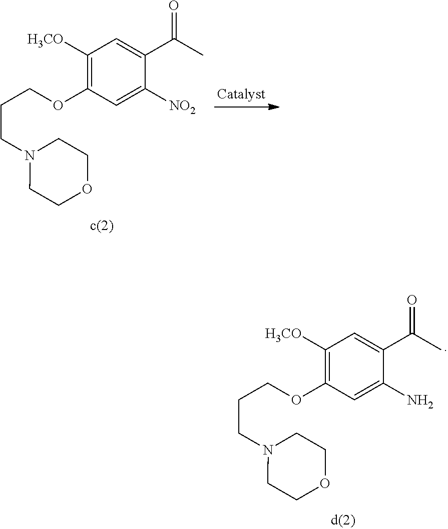 [SMILES: COc1cc(C(C)=O)c([N+](=O)[O-])cc1OCCCN1CCOCC1 ; COc1cc(C(C)=O)c(N)cc1OCCCN1CCOCC1]

The compound of formula d(2) can be in its free base form or converted to a pharmaceutically acceptable salt thereof. The reaction conditions that can be used in this aspect include any of the reaction conditions disclosed in embodiment (G) of Aspect (1).

Aspect (5) of the disclosure relates to a method of preparing a compound of formula e(2):

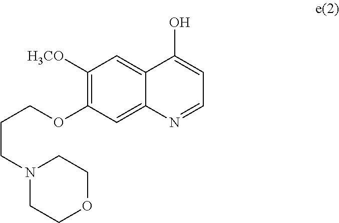 [SMILES: COc1cc2c(O)ccnc2cc1OCCCN1CCOCC1]

or a pharmaceutically acceptable salt thereof, the method comprising converting a compound of formula d(2) to a compound of formula e(2) with sodium ethoxide and an alkyl formate, such as ethyl formate, in a suitable solvent:

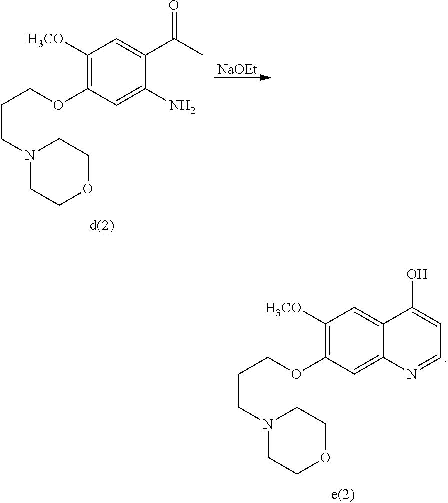 [SMILES: COc1cc(C(C)=O)c(N)cc1OCCCN1CCOCC1 ; COc1cc2c(O)ccnc2cc1OCCCN1CCOCC1 ; "NaOEt"]

The compound of formula e(2) can be in its free base form or converted to a pharmaceutically acceptable salt thereof. The reaction conditions that can be used in this aspect include any of the reaction conditions disclosed in embodiment (F) of Aspect (1).

Aspect (6) of the disclosure relates to a method of preparing a compound of formula f(2):

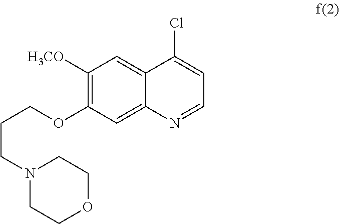 [SMILES: COc1cc2c(Cl)ccnc2cc1OCCCN1CCOCC1]

or a pharmaceutically acceptable salt thereof, the method comprising converting the compound of formula e(2) to a compound of formula f(2) with a chlorinating agent in a suitable solvent:

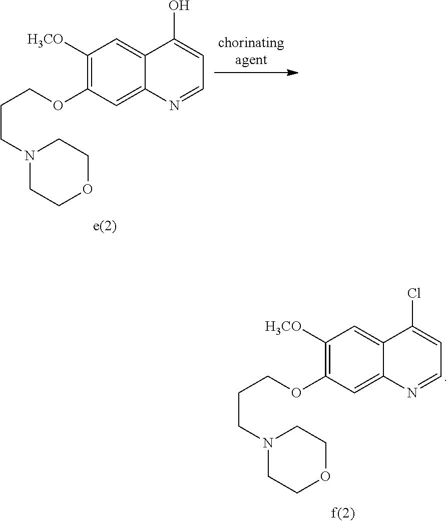 [SMILES: COc1cc2c(O)ccnc2cc1OCCCN1CCOCC1 ; COc1cc2c(Cl)ccnc2cc1OCCCN1CCOCC1]

The compound of formula f(2) can be in its free base form or converted to a pharmaceutically acceptable salt thereof. The reaction conditions that can be used in this aspect include any of the reaction conditions disclosed in embodiment (E) of Aspect (1).

Aspect (7) of the disclosure relates to a method of preparing a compound of formula g(2):

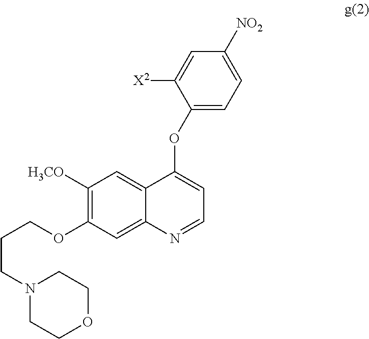

or a pharmaceutically acceptable salt thereof, the method comprising reacting the compound of formula f(2) with reactant y(2) under basic conditions in an appropriate solvent (2,6-lutidine is a sterically hindered organic mild base) to yield the compound of g(2):

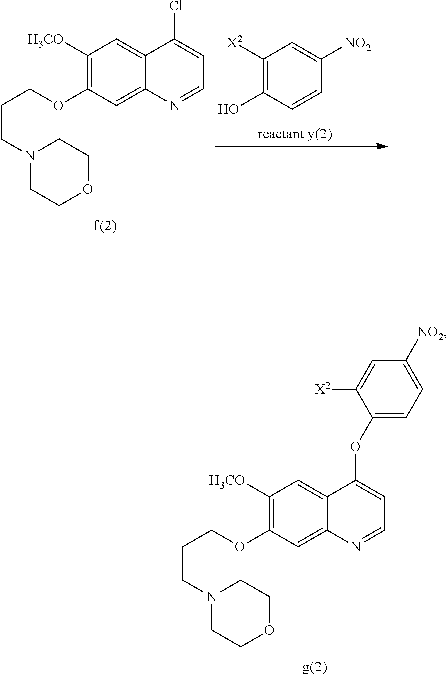

wherein X² is H, Br, Cl or F. The compound of formula g(2) can be converted to a pharmaceutically acceptable salt thereof. The reaction conditions that can be used in this aspect include any of the reaction conditions disclosed in embodiment (D) of Aspect (1).

Aspect (8) of the disclosure relates to a method of preparing a compound of formula h(2):

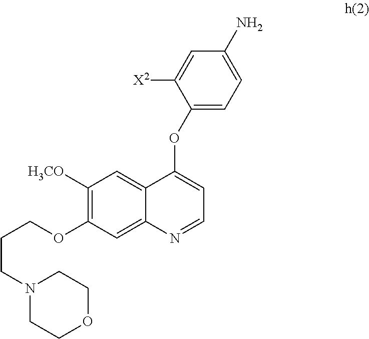

or a pharmaceutically acceptable salt thereof, the method comprising hydrogenating the compound of formula g(2) with a catalyst in a suitable solvent to yield the compound according to formula h(2):

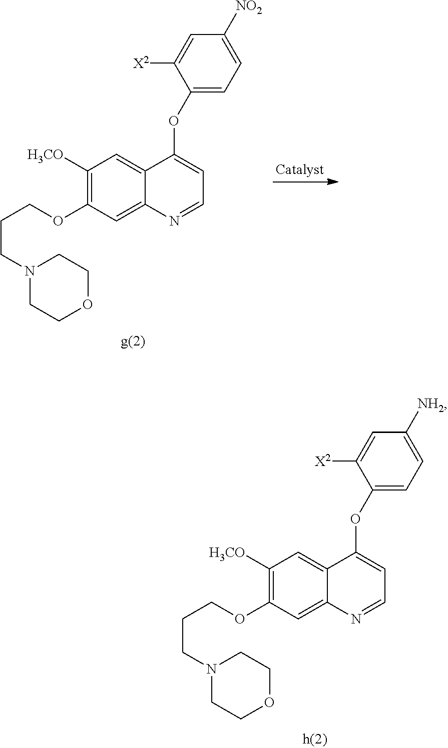

wherein X² is H, Br, Cl or F. The compound of formula h(2) can be in its free base form or converted to a pharmaceutically acceptable salt thereof. The reaction conditions that can be used in this aspect include any of the reaction conditions disclosed in embodiment (C) of Aspect (1).

Aspect (9) of the disclosure relates to a method of preparing a compound of formula (h3) by reacting the compound of formula f(3) with reactant u2 to yield the compound of formula h(3).

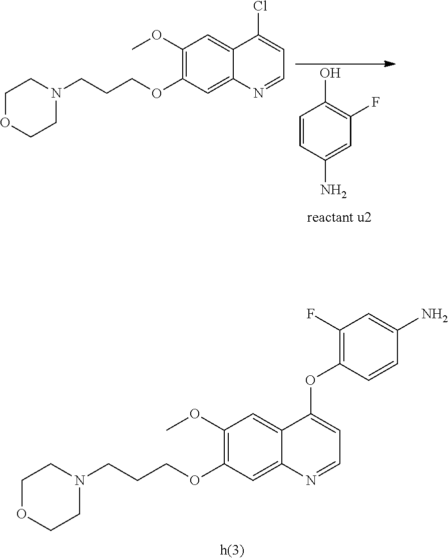

The reaction conditions that can be used in this aspect include any of the reaction conditions disclosed in alternative embodiments for embodiments (C) and (D) of Aspect (1).

### DEFINITIONS

As used in the present specification, the following words and phrases are generally intended to have the meanings as set forth below, except to the extent that the context in which they are used indicates otherwise or they are expressly defined to mean something different.

The word “can” is used in a non-limiting sense and in contradistinction to the word “must.” Thus, for example, in many aspects of the invention a certain element is described as “can” having a specified identity, which is meant to convey that the subject element is permitted to have that identity according to the invention but is not required to have it.

If a group “R” is depicted as “floating” on a ring system, then unless otherwise defined, the substituent(s) “R” can reside on any atom of the ring system, assuming replacement of a depicted, implied, or expressly defined hydrogen from one of the ring atoms, so long as a stable structure is formed.

When there are more than one such depicted “floating” groups, as for example in the formulae: where there are two groups, namely, the “R” and the bond indicating attachment to a parent structure; then, unless otherwise defined, the “floating” groups can reside on any atoms of the ring system, again assuming each replaces a depicted, implied, or expressly defined hydrogen on the ring.

Pharmaceutically acceptable salts include acid addition salts.

“Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid, and the like, or mixtures thereof, as well as organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like, or mixtures thereof.

The disclosure is further illustrated by the following examples, which are not to be construed as limiting the disclosure in scope or spirit to the specific procedures described in them.

Unless specified otherwise, the starting materials and various intermediates may be obtained from commercial sources, prepared from commercially available organic compounds, or prepared using well-known synthetic methods.

### EXPERIMENTAL PROCEDURES

The invention is illustrated further by the following examples in Scheme 1 and the description thereof, which are not to be construed as limiting the invention in scope or spirit to the specific procedures described in them. Those having skill in the art will recognize that the starting materials may be varied and additional steps employed to produce compounds encompassed by the invention, as demonstrated by the following examples. Those skilled in the art will also recognize that it may be necessary to utilize different solvents or reagents to achieve some of the above transformations.

Unless otherwise specified, all reagents and solvents are of standard commercial grade and are used without further purification. The appropriate atmosphere to run the reaction under, for example, air, nitrogen, hydrogen, argon and the like, will be apparent to those skilled in the art.

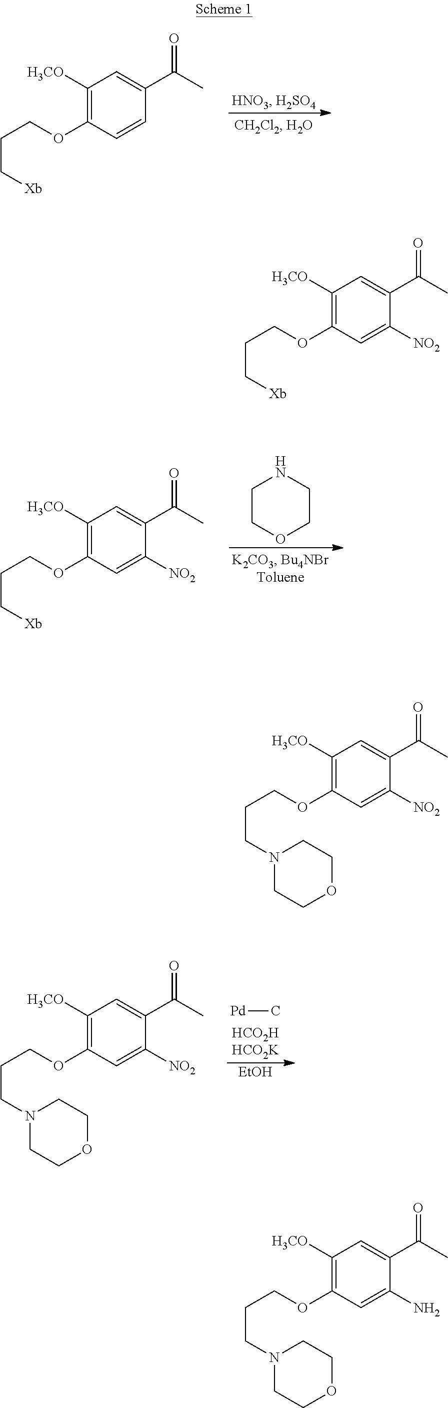

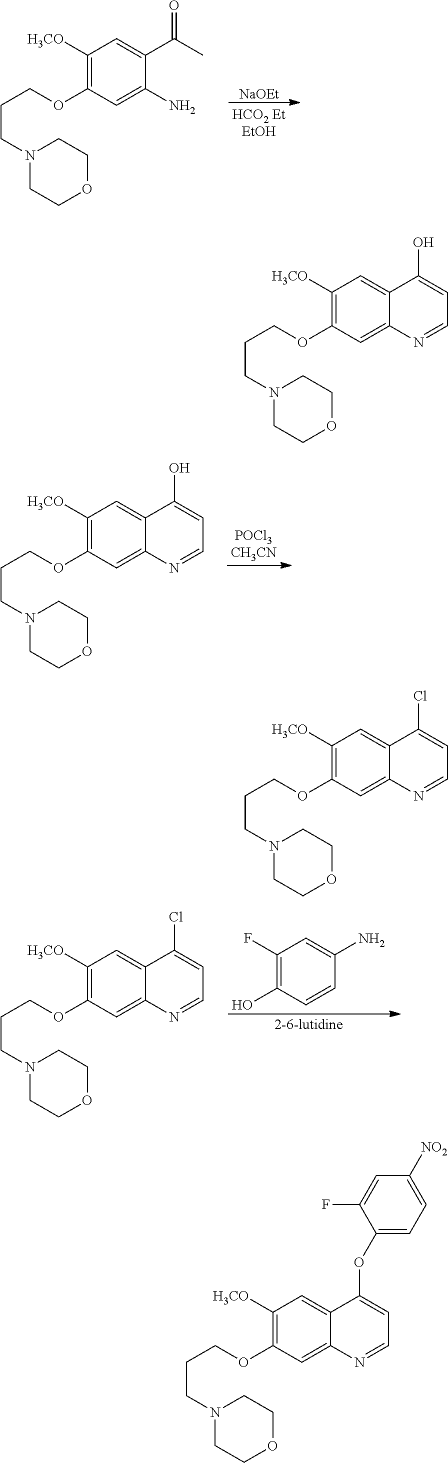

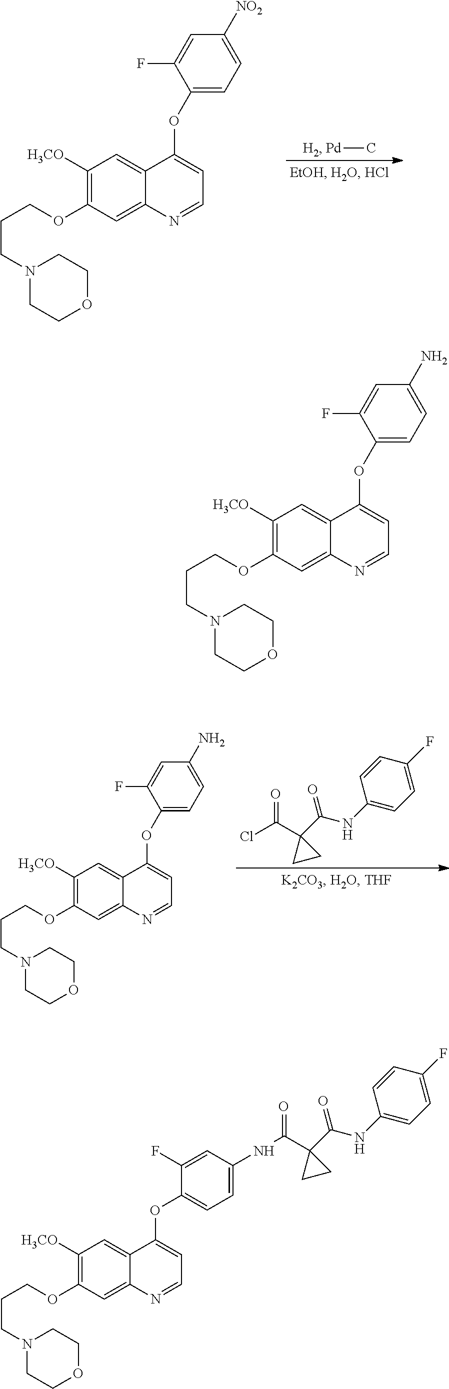

Xb in Scheme 1 above is Br or Cl. For the names of the intermediates described within the description of Scheme 1 below, Xb is referred to as halo, wherein this halo group for these intermediates is meant to mean either Br or Cl. This definition of halo, which is applicable only to these intermediates in the description of Scheme 1 below, is not meant to change the definition of halo in the definitions section.

### Preparation of 1-[5 methoxy-4 (3-halo propoxy)-2 nitro-phenyl]-ethanone

Water (70 L) was charged to the solution of 1-[4-(3-halo propoxy)-3-methoxy phenyl]ethanone (both the bromo and the chloro compound are commercially available). The solution was cooled to approximately 4° C. Concentrated sulfuric acid (129.5 kg) was added at a rate such that the batch temperature did not exceed approximately 18° C. The resulting solution was cooled to approximately 5° C. and 70% nitric acid (75.8 kg) was added at a rate such that the batch temperature did not exceed approximately 10° C. Methylene chloride, water and ice were charged to a separate reactor. The acidic reaction mixture was then added into this mixture. The methylene chloride layer was separated and the aqueous layer was back extracted with methylene chloride. The combined methylene chloride layers were washed with aqueous potassium bicarbonate solution and concentrated by vacuum distillation. 1-Butanol was added and the mixture was again concentrated by vacuum distillation. The resulting solution was stirred at approximately 20° C. during which time the product crystallized. The solids were collected by filtration, washed with 1-butanol to afford compound the title compound, which was isolated as a solvent wet cake and used directly in the next step.

¹HNMR (400 MHz, DMSO-d6): δ. 7.69 (s, 1H), 7.24 (s, 1H); 4.23 (m, 2H), 3.94 (s, 3H), 3.78 (t)-3.65 (t) (2H), 2.51 (s, 3H), 2.30-2.08 (m, 2H) LC/MS Calcd for [M(Cl)+H]⁺ 288.1. found 288.0; Calcd for [M(Br)+H]⁺ 332.0, 334.0. found 331.9, 334.0.

### Preparation of 1-[5-methoxy-4-(3-morpholin-4-yl-propoxy)-2-nitro-phenyl]-ethanone

The solvent wet cake isolated in the previous step was dissolved in toluene. A solution of sodium iodide (67.9 kg) and potassium carbonate (83.4 kg) was added to this solution, followed by tetrabutylammonium bromide (9.92 kg) and morpholine (83.4 kg). The resulting 2 phase mixture was heated to approximately 85° C. for about 9 hours. The mixture was then cooled to ambient temperature. The organic layer was removed. The aqueous layer was back extracted with toluene. The combined toluene layers were washed sequentially with two portions of saturated aqueous sodium thiosulfate followed by two portions of water. The resulting solution of the title compound was used in the next step without further processing.

¹HNMR (400 MHz, DMSO-d6): δ. 7.64 (s, 1H), 7.22 (s, 1H), 4.15 (t, 2H), 3.93 (s, 3H), 3.57 (t, 4H), 2.52 (s, 3H), 2.44-2.30 (m, 6H), 1.90 (quin, 2H); LC/MS Calcd for [M+H]⁺ 339.2. found 339.2.

### Preparation of 1-[2-amino-5-methoxy-4-(3-morpholin-4-yl-propoxy)-phenyl]-ethanone

The solution from the previous step was concentrated under reduced pressure to approximately half of the original volume. Ethanol and 10% Pd C (50% water wet, 5.02 kg) were added; the resulting slurry was heated to approximately 48° C. and an aqueous solution of formic acid (22.0 kg) and potassium formate (37.0 kg) was added. When the addition was complete and the reaction deemed complete by thin layer chromatography (TLC), water was added to dissolve the by-product salts. The mixture was filtered to remove the insoluble catalyst. The filtrate was concentrated under reduced pressure and toluene was added. The mixture was made basic (pH of about 10) by the addition of aqueous potassium carbonate. The toluene layer was separated and the aqueous layer was back extracted with toluene. The combined toluene phases were dried over anhydrous sodium sulfate. The drying agent was removed by filtration and the resulting solution was used in the next step without further processing.

¹HNMR (400 MHz, DMSO-d6): δ. 7.11 (s, 1H), 7.01 (br s, 2H), 6.31 (s, 1H), 3.97 (t, 2H), 3.69 (s, 3H), 3.57 (t, 4H), 2.42 (s, 3H), 2.44-2.30 (m, 6H), 1.91 (quin, 2H LC/MS Calcd for [M+H]⁺ 309.2. found 309.1.

### Preparation of 6-methoxy-7-(3-morpholin-4-yl-propoxy)-quinolin-4-ol, sodium salt

A solution of sodium ethoxide (85.0 kg) in ethanol and ethyl formate (70.0 kg) was added to the solution from the previous step. The mixture was warmed to approximately 44° C. for about 3 hours. The reaction mixture was cooled to approximately 25° C. Methyl t-butyl ether (MTBE) was added which caused the product to precipitate. The product was collected by filtration and the cake was washed with MTBE and dried under reduced pressure at ambient temperature. The dried product was milled through a mesh screen to afford 60.2 kg of the title compound.

¹HNMR (400 MHz, DMSO-d6): δ. 11.22 (br s, 1H), 8.61 (d, 1H), 7.55 (s, 1H), 7.54 (s, 1H), 7.17 (d, 1H), 4.29 (t, 2H), 3.99 (m, 2H), 3.96 (s, 3H), 3.84 (t, 2H), 3.50 (d, 2H), 3.30 (m, 2H), 3.11 (m, 2H), 2.35 (m, 2H), LC/MS Calcd for [M+H]⁺ 319.2. found 319.1.

### Preparation of 4-chlor-6-methoxy-7-(3 morpholin-4-yl)-quinoline

Phosphorous oxychloride (26.32 kg) was added to a solution of 6-methoxy-7-(3-morpholin-4-yl-propoxy)-quinolin-4-ol (5.00 kg) in acetonitrile that was heated to 50-55° C. When the addition was complete, the mixture was heated to reflux (approximately 82° C.) and held at that temperature, with stirring for approximately 18 hours at which time it was sampled for in process HPLC analysis. The reaction was considered complete when NMT 5% starting material remained. The reaction mixture was then cooled to 20-25° C. and filtered to remove solids. The filtrate was then concentrated to a residue. Acetronitrile was added and the resulting solution was concentrated to a residue. Methylene chloride was added to the residue and the resulting solution was quenched with a mixture of methylene chloride and aqueous ammonium hydroxide. The resulting 2 phase mixture was separated and the aqueous layer was back extracted with methylene chloride. The combined methylene chloride solutions were dried over anhydrous magnesium sulfate, filtered and concentrated to a solid. The solids were dried at 30-40° C. under reduced pressure to afford the title compound (1.480 kg).

¹HNMR (400 MHz, DMSO-d6): δ. 8.61 (d, 1H), 7.56 (d, 1H), 7.45 (s, 1H), 7.38 (s, 1H), 4.21 (t, 2H), 3.97 (s, 3H), 3.58 (m, 2H), 2.50-2.30 (m, 6H), 1.97 (quin, 2H) LC/MS Calcd for [M+H]⁺458.2. found 458.0.

### Preparation of 4-(2-fluoro-4-nitro-phenoxy)-6-methoxy-7-(3-morpholin-4-yl propoxy)quinoline

A solution of 4-chloro-6-methoxy-7-(3 morpholin-4-yl)-quinoline (2.005 kg, 5.95 mol) and 2 fluoro-4-nitrophenol (1.169 kg, 7.44 mol) in 2,6-lutidine was heated to 140-145° C., with stirring, for approximately 2 hours, at which time it was sampled for in process HPLC analysis. The reaction was considered complete when less than 5% starting material remained. The reaction mixture was then cooled to approximately 75° C. and water was added. Potassium carbonate was added to the mixture, which was then stirred at ambient temperature overnight. The solids that precipitated were collected by filtration, washed with aqueous potassium carbonate, and dried at 55-60° C. under reduced pressure to afford the title compound (1.7 kg).

¹HNMR (400 MHz, DMSO-d6): δ 8.54 (d, 1H), 8.44 (dd, 1H), 8.18 (m, 1H), 7.60 (m, 1H), 7.43 (s, 1H), 7.42 (s, 1H), 6.75 (d, 1H), 4.19 (t, 2H), 3.90 (s, 3H), 3.56 (t, 4H), 2.44 (t, 2H), 2.36 (m, 4H), 1.96 (m, 2H). LC/MS Calcd for [M+H]⁺ 337.1, 339.1. found 337.0, 339.0.

### Preparation of 3-fluoro-4-[6-methoxy-7-(3-morpholin-4-yl-propoxy)-quinolin-4-yloxy]-phenylamine

A reactor containing 4-(2-fluoro-4-nitro-phenoxy)-6-methoxy-7-(3-morpholin-4-yl propoxy)quinoline (2.5 kg) and 10% palladium on carbon (50% water wet, 250 g) in a mixture of ethanol and water containing concentrated hydrochloric acid (1.5 L) was pressurized with hydrogen gas (approximately 40 psi). The mixture was stirred at ambient temperature. When the reaction was complete (typically 2 hours), as evidenced by in process HPLC analysis, the hydrogen was vented and the reactor inerted with argon. The reaction mixture was filtered through a bed of Celite® to remove the catalyst. Potassium carbonate was added to the filtrate until the pH of the solution was approximately 10. The resulting suspension was stirred at 20-25° C. for approximately 1 hour. The solids were collected by filtration, washed with water and dried at 50-60° C. under reduced pressure to afford the title compound (1.164 kg).

¹H NMR (400 MHz, DMSO-d6): δ 8.45 (d, 1H), 7.51 (s, 1H), 7.38 (s, 1H), 7.08 (t, 1H), 6.55 (dd, 1H), 6.46 (dd, 1H), 6.39 (dd, 1H), 5.51 (br. s, 2H), 4.19 (t, 2H), 3.94 (s, 3H), 3.59 (t, 4H), 2.47 (t, 2H), 2.39 (m, 4H), 1.98 (m, 2H). LC/MS Calcd for [M+H]⁺428.2. found 428.1

### Preparation of 1-(4-fluoro-phenylcarbamoyl)-cyclopropanecarboxylic acid

Triethylamine (7.78 kg) was added to a cooled (approximately 4° C.) solution of commercially available cyclopropane 1,1-dicarboxylic acid (9.95 kg) in THF, at a rate such that the batch temperature did not exceed 10° C. The solution was stirred for approximately 30 minutes and then thionyl chloride (9.14 kg) was added, keeping the batch temperature below 10° C. When the addition was complete, a solution of 4 fluoroaniline (9.4 kg) in THF was added at a rate such that the batch temperature did not exceed 10° C. The mixture was stirred for approximately 4 hours and then diluted with isopropyl acetate. The diluted solution was washed sequentially with aqueous sodium hydroxide, water, and aqueous sodium chloride. The organic solution was concentrated by vacuum distillation. Heptane was added to the concentrate. The resulting slurry was filtered by centrifugation and the solids were dried at approximately 35° C. under vacuum to afford the title compound (10.2 kg).

¹H NMR (400 MHz, DMSO-d6): 13.06 (br s, 1H), 10.58 (s, 1H), 7.65-7.60 (m, 2H), 7.18-7.12 (m, 2H), 1.41 (s, 4H), LC/MS Calcd for [M+H]⁺ 224.1. found 224.0

### Preparation of 1-(4-fluoro-phenylcarbamoyl)-cyclopropanecarbonylchloride

Oxalyl chloride (291 mL) was added slowly to a cooled (approximately 5° C.) solution of 1-(4-fluoro-phenylcarbamoyl)-cyclopropanecarboxylic acid in THF at a rate such that the batch temperature did not exceed 10° C. When the addition was complete, the batch was allowed to warm to ambient temperature and held with stirring for approximately 2 hours, at which time in process HPLC analysis indicated the reaction was complete. The solution was used in the next step without further processing.

### Preparation of cyclopropane-1,1-dicarboxylic acid {3-fluoro-4-[6-methoxy-7-(3-morpholin-4-yl-propoxy)-quinolin-4-ylamino]phenyl}-amide-(4 fluorophenyl)-amide

The solution from the previous step was added to a mixture of 3-fluoro-4-[6-methoxy-7-(3-morpholin-4-yl-propoxy)-quinolin-4-yloxy]-phenylamine (1160 kg) and potassium carbonate (412.25 g) in THF and water at a rate such that the batch temperature was maintained at approximately 15-21° C. When the addition was complete, the batch was warmed to ambient temperature and held with stirring for approximately 1 hour, at which time in process HPLC analysis indicated the reaction was complete. Aqueous potassium carbonate solution and isopropyl acetate were added to the batch. The resulting 2-phase mixture was stirred and then the phases were allowed to separate. The aqueous phase was back extracted with isopropyl acetate. The combined isopropyl acetate layers were washed with water followed by aqueous sodium chloride and then slurried with a mixture of magnesium sulfate and activated carbon. The slurry was filtered over Celite® and the filtrate was concentrated to an oil at approximately 30° C. under vacuum to afford the title compound which was carried into the next step without further processing.

¹H NMR (400 MHz, DMSO-d6): δ 10.41 (s, 1H), 10.03 (s, 1H), 8.47 (d, 1H), 7.91 (dd, 1H), 7.65 (m, 2H), 7.53 (m, 2H), 7.42 (m, 2H), 7.16 (t, 2H), 6.41 (d, 1H), 4.20 (t, 2H), 3.95 (s, 3H), 3.59 (t, 4H), 2.47 (t, 2H), 2.39 (m, 4H), 1.98 (m, 2H), 1.47 (m, 4H). LC/MS Calcd for [M+H]⁺ 633.2. found 633.1

### Preparation of the bisphosphate salt of cyclopropane-1,1-dicarboxylic acid {3-fluoro-4-[6-methoxy-7-(3-morpholin-4-yl-propoxy)-quinolin-4-ylamino]phenyl}-amide (4-fluoro-phenyl)-amide

Cyclopropane-1,1-dicarboxylic acid {3-fluoro-4-[6-methoxy-7-(3-morpholin-4-yl-propoxy)-quinolin-4-ylamino]phenyl}amide-(4 fluoro phenyl)-amide from the previous step was dissolved in acetone and water. Phosphoric acid (85%, 372.48 g) was added at a rate such that the batch temperature did not exceed 30° C. The batch was maintained at approximately 15-30° C. with stirring for 1 hour during which time the product precipitated. The solids were collected by filtration, washed with acetone and dried at approximately 60° C. under vacuum to afford the title compound (1.533 kg). The title compound has a c-Met IC₅₀ value of less than 50 nM. The bisphosphate salt is not shown in scheme 1.

¹H NMR (400 MHz, DMSO-d6): (diphosphate) 10.41 (s, 1H), 10.02 (s, 1H), 8.48 (d, 1H), 7.93 (dd, 1H), 7.65 (m, 2H), 7.53 (d, 2H), 7.42 (m, 2H), 7.17 (m, 2H), 6.48 (d, 1H), 5.6 (br s, 6H), 4.24 (t, 2H), 3.95 (s, 3H), 3.69 (bs, 4H), 2.73 (bs, 6H), 2.09 (t, 2H), 1.48 (d, 4H).

### Procedure for Direct Coupling

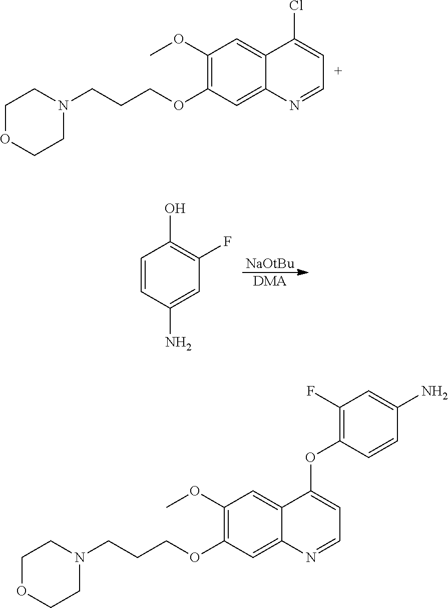

Solid sodium tert-butoxide (1.20 g; 12.5 mmol) was added to a suspension of the chloroquinoline (3.37 g; 10 mmol) in dimethylacetamide (35 mL), followed by solid 2-fluoro-4-hydroxyaniline. The dark green reaction mixture was heated at 95-100° C. for 18 h. HPLC analysis showed approximately. 18% starting material remaining and ca. 79% product. The reaction mixture was cooled to below 50° C. and additional sodium tert-butoxide (300 mg; 3.125 mmol) and aniline (300 mg; 2.36 mmol) were added and heating at 95-100° C. was resumed. HPLC analysis after 18 h revealed less than 3% starting material remaining. The reaction was cooled to below 30° C., and ice water (50 mL) was added while maintaining the temperature below 30° C. After stirring for 1 hour at room temperature, the product was collected by filtration, washed with water (2×10 mL) and dried under vacuum on the filter funnel, to yield 4.11 g of the coupled product as a tan solid (96% yield; 89%, corrected for water content).

¹H NMR and MS: consistent with product; 97.8% LCAP; ˜7 wt % water by KF.

The foregoing disclosure has been described in some detail by way of illustration and example, for purposes of clarity and understanding. The invention has been described with reference to various specific and preferred embodiments and techniques. However, it should be understood that many variations and modifications can be made while remaining within the spirit and scope of the invention. It will be obvious to one of skill in the art that changes and modifications can be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

1. A method of preparing a compound of formula i(1): 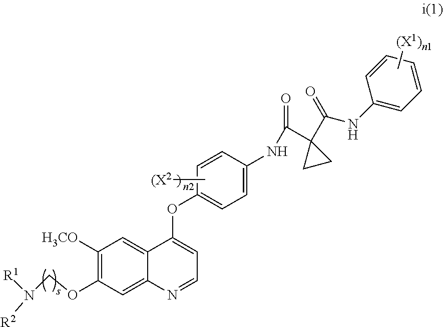
or a pharmaceutically acceptable salt thereof, wherein:
R¹ and R² join together with the nitrogen atom to which they are attached to form a 6 membered heterocycloalkyl;
X¹ is H, Br, Cl or F;
X² is H, Br, Cl or F;
s is 2-6;
n1 is 1-2; and
n2 is 1-2,
the method comprising:
contacting the compound of formula h(1) with reactant z(1) to yield the compound of formula i(1):

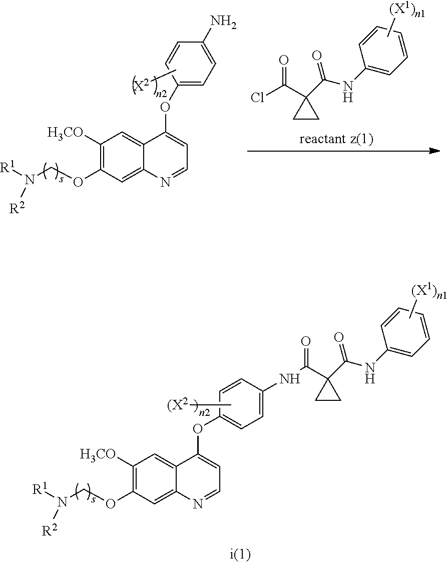

2. The method according to claim 1, wherein s is 3; and R¹ and R² join together with the nitrogen atom to which they are attached to form a morpholinyl.

3. The method according to claim 1, wherein the compound of formula h(1) is made by reducing a compound of formula g(1) to yield the compound of formula h(1): 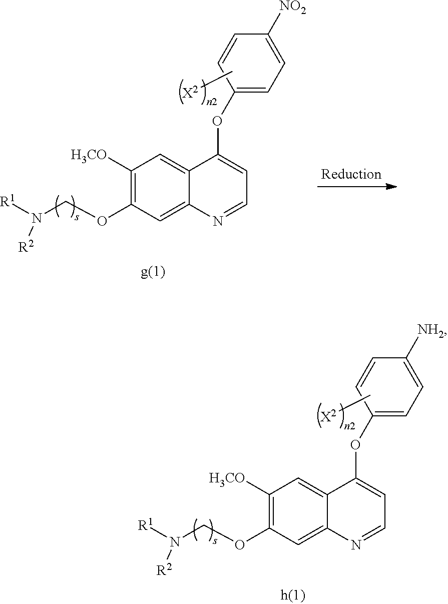
wherein each of R¹, R², X², S and n2 are as defined in claim 1.

4. The method according to claim 1, wherein the compound of formula h(1) is made by reacting a compound of formula f(1) with reactant u to yield the compound of formula h(1): 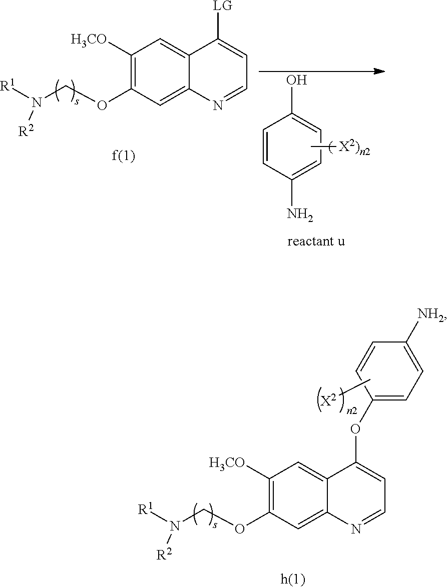
wherein LG represents a leaving group.

5. The method according to claim 3, wherein the compound of formula g(1) is made by reacting a compound of formula f(1) with reactant y(1) to yield the compound of formula g(1): 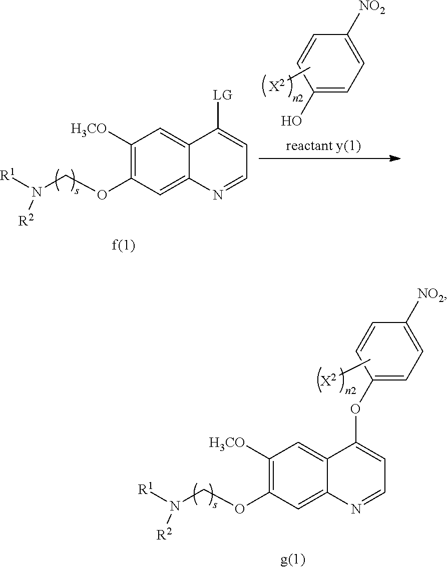
wherein LG represents a leaving group, and each of R¹, R², X², s and n2 are as defined in claim 1.

6. The method according to claim 5, wherein the compound of formula f(1) is made by converting a compound of formula e(1) to the compound of formula f(1): 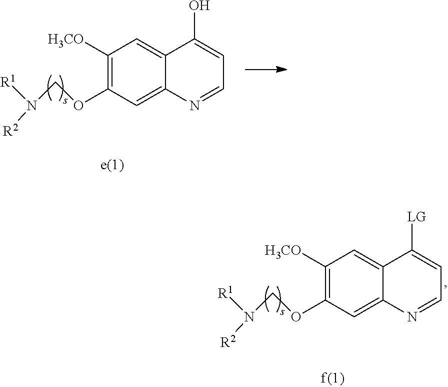
wherein LG represents a leaving group, and each of s, R¹ and R² are as defined in claim 1.

7. The method according to claim 6, wherein the compound of formula e(1) is made by converting a compound of formula d(1) to the compound of formula e(1) with an alkyl formate: 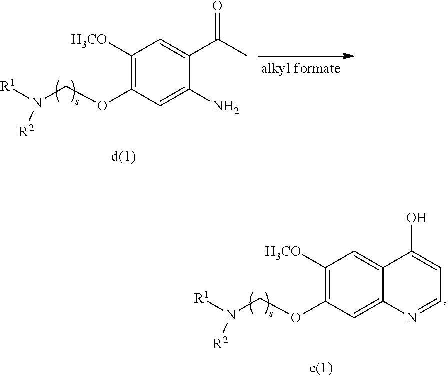
wherein each of s, R¹ and R² are as defined in claim 1.

8. The method according to claim 7, wherein the compound of formula d(1) is made by reducing a compound of formula c(1) to yield the compound of formula d(1): 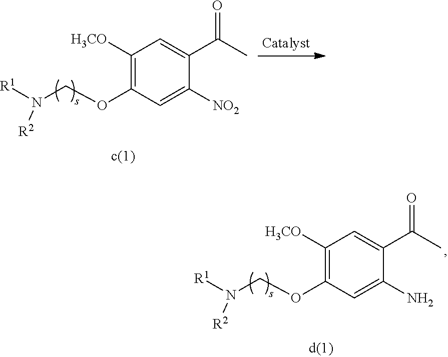
wherein each of s, R¹ and R² are as defined in claim 1.

9. The method according to claim 8, wherein the compound of formula c(1) is made by reacting the compound of formula b(1) with 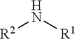
to yield the compound of formula c(1): 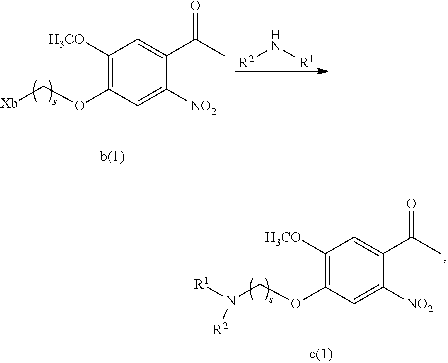
wherein Xb is Br or Cl; and each of s, R¹ and R² are as defined in claim 1.

10. The method according to claim 9, wherein the compound of formula b(1) is made by reacting a compound of formula a(1) with HNO₃ to yield the compound of formula b(1): 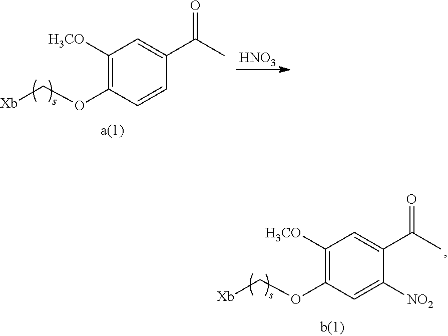
wherein Xb is Br or Cl; and each of s, R¹ and R² are as defined in claim 1.

11. The method according to claim 1, wherein reactant z(1) is made by reacting reactant z(1a) with a chlorinating agent to yield reactant z(1): 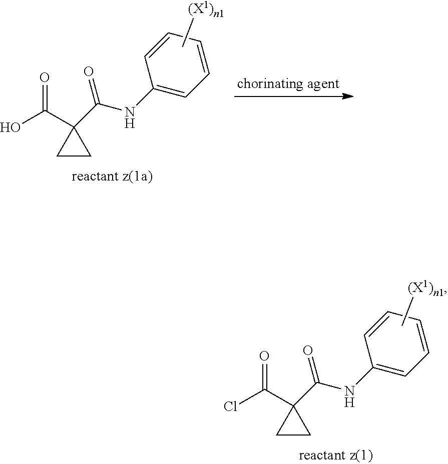
wherein X¹ is Br, Cl or F; and n1 is 1-2.

12. The method according to claim 1, wherein the compound of formula i(1) is of formula i(2): 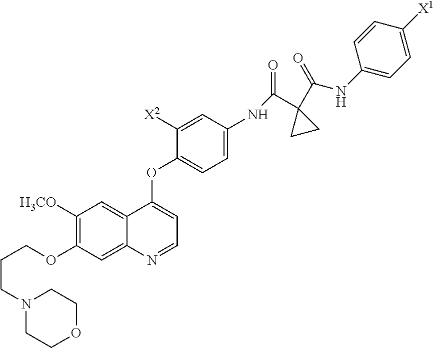
or a pharmaceutically acceptable salt thereof, wherein:
X¹ is H, Cl, Br or F; and
X² is H, Cl, Br or F.

13. The method according to claim 5, wherein the compound of formula f(1) is of formula f(2): 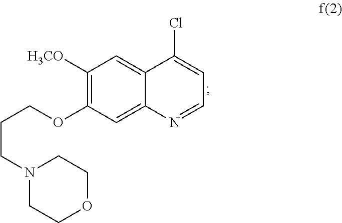 [SMILES: COc1cc2c(Cl)ccnc2cc1OCCCN1CCOCC1]
reactant y(1) is reactant (y)(2): 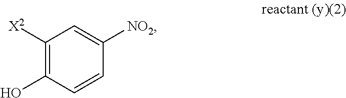
wherein X² is chloro or fluoro; and
the compound of formula g(1) is of formula g(2):

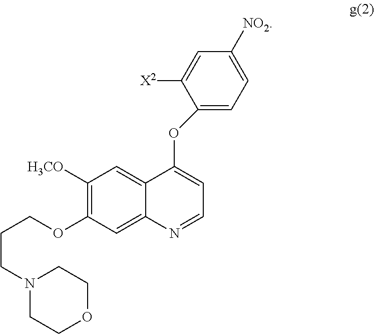

14. The method according to claim 3, wherein the compound of formula g(1) is of formula g(2): 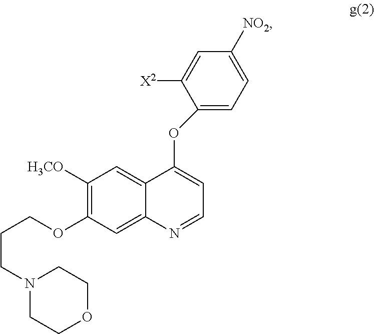
wherein X² is chloro or fluoro; and
the compound of formula h(1) is of formula h(2):

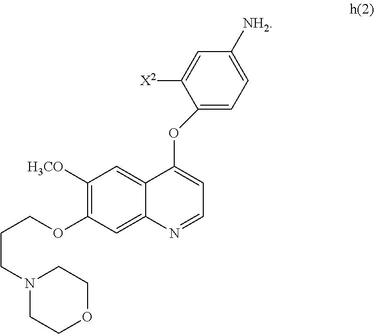

15. The method according to claim 1, wherein the compound of formula h(1) is of formula h(2): 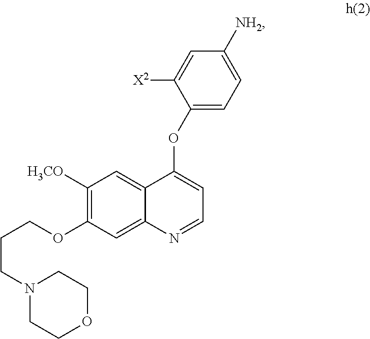
wherein X² is F;
reactant z(1) is reactant (z)(2): 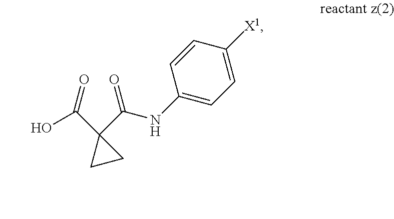
wherein X¹ is F; and
the compound of formula i(1) is of formula i(2):

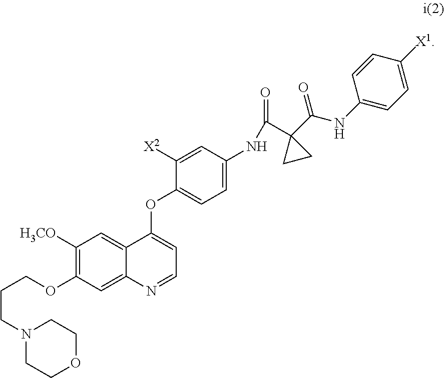

16. The method according to claim 15, wherein the compound of formula i(2) is: 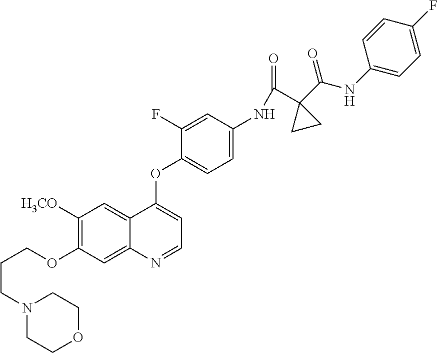 [SMILES: COc1cc2c(Oc3ccc(NC(=O)C4(C(=O)Nc5ccc(F)cc5)CC4)cc3F)ccnc2cc1OCCCN1CCOCC1]
or a pharmaceutically acceptable salt thereof.

17. The method according to claim 16, wherein the compound of formula i(2) is the bis-phosphate or bis-maleate salt.
